(19) United States
(12) Patent Application Publication
(10) Pub. No.: US 2021/0308400 A1
(43) Pub. Date: Oct. 7, 2021

(54) SOFTWARE-DEFINED VENTILATOR PLATFORM

(71) Applicant: OPTICAL ENGINES, INC., Colorado Springs, CO (US)

(72) Inventors: Donald Lee Sipes, JR., Colorado Springs, CO (US); Matthew Schulz, Colorado Springs, CO (US)

(21) Appl. No.: 17/223,518

(22) Filed: Apr. 6, 2021

Related U.S. Application Data
(60) Provisional application No. 63/005,693, filed on Apr. 6, 2020.

(51) Int. Cl. A61M 16/00; A61M 16/10; A61M 16/20

(52) U.S. Cl. CPC A61M 16/024; A61M 16/0066; A61M 16/1055; A61M 16/201; A61M 2205/3584; A61M 2202/0208; A61M 2205/3327; A61M 2016/0027; A61M 2016/003; A61M 2205/502

(57) ABSTRACT

A ventilator system includes: a control system having a controller and a user interface; and a pneumatic system having an inspiratory channel and an expiratory channel. The inspiratory channel further comprises a blower connected to an oxygen source, wherein the blower is configured to be controlled by the controller, and to deliver oxygen and/or air to a patient via the inspiratory channel. The expiratory channel includes an exhaust valve configured to be controlled by the controller and an outlet. The pneumatic system further includes a plurality of sensors configured to provide oxygen measurements, pressure measurements, and flow measurements. The controller is configured to receive a user input via the user interface for controlling a treatment regimen for the patient, and to control the blower and the exhaust valve according to the treatment regimen.

### CROSS-REFERENCE TO RELATED APPLICATION

This patent application claims the benefit of U.S. Provisional Patent Application No. 63/005,693, filed on Apr. 6, 2020, which is incorporated by reference herein in its entirety.

### BACKGROUND

In the time of coronavirus (COVID), healthcare systems across the country and around the globe have needed to cope with extremely heavy workloads and were close to reaching a breaking point. Not only were there not enough healthcare providers available to provide care, the necessary equipment was also scarce. Many patients with relatively severe cases of COVID needed ventilators to provide critical breath support, but with the fast rise of the infection numbers, there were not enough ventilators available to accommodate the large numbers of hospitalized patients needing such breath support.

Additionally, there were not any established treatment protocols for patients whose symptoms are not severe enough for hospital admission. For many COVID sufferers, there was uncertainty as to whether they should stay at home or go to the hospital. And for patients who went to the hospital only to be turned away and sent home, such patients often ended up waiting at home too long and did not go back to the hospital until their health had substantially degraded. Better outcomes would have been achievable for many such patients if they had been able to receive breath support at an earlier stage.

Part of the problem is that existing ventilators are very complex and costly. Ventilators currently used in hospital settings are very sophisticated, highly specialized, and feature-rich machines. These ventilators are composed of custom-built boards with embedded processors, analog sensors, and valves, and they provide health care providers with the ability to “design the breadth” of their most seriously ill, intensive care patients. For example, these ventilators have advanced diagnostic algorithms for real-time assessment of the patient's lung function and can precisely monitor and control pressure and flow rate every few hundredths of a second, which enables the ventilators to constantly and automatically adapt to what each patient needs. However, because of their capabilities, such ventilators are very complicated medical machines and require a large number of customized parts, which are built in relatively small volumes. This causes the production to be slow and the cost to be high. Additionally, health care providers, including doctors, respirator technicians, and nurses, need to be trained to understand the key functions of these complicated ventilators, as well as how to operate them.

On the other end of the spectrum, simple breath support machines exist as well. For example, there are manual resuscitators which require manual squeezing of a bag to provide air or control and monitoring of airflow through manual knobs and liquid-crystal display (LCD) displays. However, these simple breath support machines are not able to provide the level of breath support needed for COVID patients, and thus would not be as effective as high-performance ventilators in improving patient outcomes for COVID cases (and other severe respiratory diseases) through early breath support intervention. Moreover, because of the simplicity of such devices, the device operator (which may have to be the patients themselves in an at-home care scenario) would need substantial operator training as to how to use the device and be kept up to date as to new treatment protocols. Further, the device operator (which may be a healthcare provider in certain scenarios) needs to devote more time and effort to operating the device, which may not be possible in a crisis situation in which healthcare providers are stretched thin.

### SUMMARY

In an exemplary embodiment, the present invention provides a ventilator system. The ventilator system includes: a control system having a controller and a user interface; and a pneumatic system having an inspiratory channel and an expiratory channel. The inspiratory channel further comprises a blower connected to an oxygen source, wherein the blower is configured to be controlled by the controller, and to deliver oxygen and/or air to a patient via the inspiratory channel. The expiratory channel includes an exhaust valve configured to be controlled by the controller and an outlet. The pneumatic system further includes a plurality of sensors configured to provide oxygen measurements, pressure measurements, and flow measurements. The controller is configured to receive measurements from the plurality of sensors and to cause the measurements to be displayed via the user interface. The controller is configured to receive a user input via the user interface for controlling a treatment regimen for the patient, and to control the blower and the exhaust valve according to the treatment regimen.

The pneumatic system may further include a negative pressure channel comprising a second blower and a second exhaust, wherein the second blower is configured to blow air out through the second exhaust to maintain a negative pressure environment proximate to a patient's face or head.

The pneumatic system may further include a biofilter disposed in the negative pressure channel for blocking transmission of viral or bacterial particles.

The pneumatic system may further include a motor controller/driver connected to the blower, and the controller may be configured to communicate with the motor controller/driver to set a revolution rate for a motor of the blower.

The plurality of sensors may include an oxygen sensor disposed in the inspiratory channel, one or more pressure sensors disposed in the inspiratory and/or expiratory channels, a first flow meter disposed in the inspiratory channel, and a second flow meter disposed in the expiratory channel.

The plurality of sensors may include a first combined flow/pressure sensing device in the inspiratory channel and a second combined flow/pressure sensing device in the expiratory channel.

The plurality of sensors may further include a carbon dioxide sensor disposed in the expiratory channel.

The pneumatic system may further include biofilters disposed in the inspiratory and expiratory channels for blocking transmission of viral or bacterial particles.

The inspiratory channel may further include an inspiratory hold valve, and the controller may be configured to control the exhaust valve and the inspiratory hold valve to perform an inspiratory hold operation, wherein during the inspiratory hold operation, both the exhaust valve and the inspiratory hold valve are closed.

The controller may be configured to communicate with a nurse call system.

The controller may be configured to trigger a nurse call to the nurse call system based on determining that one or more measurements from the plurality of sensors satisfies nurse call criteria.

The controller may be configured to communicate over a communication network and to be remotely controlled by a healthcare provider device via the communication network.

The user interface may include a video camera, and the controller may be configured to provide telemedicine communications between a patient and a healthcare provider via the video camera.

The controller may be configured to obtain flow measurements from a proximal flow sensor disposed in a tubing circuit connected to the pneumatic system, wherein the proximal flow sensor is located proximate to a patient's face or airway.

The controller may be configured to operate the pneumatic system in a plurality of modes corresponding to respective treatment regimens, wherein each mode comprises a respective set of adjustable parameters.

The plurality of modes may include a continuous positive airway pressure (CPAP) mode, a biphasic positive airway pressure (BiPAP) mode, a pressure controlled inverse ratio ventilation (PC-IRV) mode, an airway pressure release ventilation (APRV) mode, a spontaneous (SPONT) mode, a pressure controlled continuous mandatory ventilation (P-CMV) mode, a pressure controlled synchronized intermittent mandatory ventilation (P-SIMV) mode, a volume controlled continuous mandatory ventilation (V-CMV) mode, and a volume controlled synchronized intermittent mandatory ventilation (V-SIMV) mode.

In another exemplary embodiment, the present invention provides a ventilator system, comprising: a control system having a controller and a user interface; and a pneumatic system having an inspiratory channel and an expiratory channel. The inspiratory channel further comprises a blower connected to an oxygen source, wherein the blower is configured to be controlled by the controller, and to deliver oxygen and/or air to a patient via the inspiratory channel. The expiratory channel includes an exhaust valve configured to be controlled by the controller and an outlet. The pneumatic system further includes a plurality of sensors configured to provide oxygen measurements, pressure measurements, and flow measurements. The controller is configured to operate the pneumatic system in a plurality of modes corresponding to respective treatment regimens, wherein each mode comprises a respective set of adjustable parameters.

The controller may be further configured to automatically adjust respective parameters based on measurements from the plurality of sensors.

The controller may be configured to adjust at least the following parameters: continuous positive airway pressure; breath rate; flow trigger; oxygen percentage; inspiratory pressure to deliver; expiratory pressure to deliver; expiratory to inspiratory ratio or inspiratory to expiratory ratio; positive expiratory end pressure (PEEP); high pressure value to deliver during inspiration; pressure to deliver when the patient initiates a breath; expiratory trigger sensitivity; and volume of air to deliver with each breath.

The controller may be configured to adjust a respective parameter with a respective mode according to a respective value range corresponding to the respective parameter for the respective mode.

In other exemplary embodiments, the present invention may provide other systems, methods, devices, non-transitory computer-readable mediums, etc. in accordance with the principles discussed in the detailed description below.

### BRIEF DESCRIPTION OF THE DRAWINGS

FIG. 1 is a block diagram depicting a software-defined ventilator system according to an exemplary embodiment of the present application:

FIG. 2 is a schematic diagram depicting a pneumatic system of a software-defined ventilator system according to an exemplary embodiment of the present application;

FIG. 3 is a schematic/block diagram depicting a test environment for a software-defined ventilator system according to an exemplary embodiment of the present application;

FIG. 4 is a schematic/block diagram depicting a treatment environment for a software-defined ventilator system according to an exemplary embodiment of the present application;

FIGS. 5A-5B are exemplary flowcharts depicting a process for operating a software-defined ventilator system according to an exemplary embodiment of the present application;

FIGS. 6A-6L are exemplary illustrations depicting user interfaces of a software-defined ventilator system according to an exemplary embodiment of the present application; and

FIG. 7 is an exemplary flowchart depicting a process for performing breath loops in a P-CMV mode of a software-defined ventilator system according to an exemplary embodiment.

### DETAILED DESCRIPTION

Exemplary embodiments of the present application provide a hardware-enabled and software-controlled ventilator that can flexibly accommodate a wide range of breath support operations using a standardized, low-cost pneumatic system. The ventilator is also referred to as a “software-defined ventilator platform” herein because the pneumatic system supports a variety of ventilator operations which can be controlled through software, whereby the software is easily reconfigurable to incorporate updated treatment regimens and protocols. The software-defined ventilator platform of exemplary embodiments of the present application is easier to manufacture, much less costly, and more user-friendly than existing high performance ventilators, and provides enhanced functionality relative to existing manual resuscitators in a manner that allows for improved patient outcomes (e.g., for early intervention with respect to COVID patients or patients having other health issues for which breath support is beneficial). Other advantages of the software-defined ventilator platform according to exemplary embodiments of the present application include: reliability, ruggedness, lower maintenance requirements, and flexibility to easily incorporate future innovations and adapt to future protocols.

Exemplary embodiments of the software-defined ventilator platform described herein may replace high-end ventilators in some applications in which high-end ventilators are currently used, but there may be certain applications where a conventional high-end ventilator may still be needed (for example, when the degree of precision with respect to oxygen control requires specialized hardware to achieve such precision). Use cases for exemplary embodiments of the software-defined ventilator platform described herein include treatment of patients at home, treatment of patients on-the-go (for example, the software-defined ventilator platform may be incorporated into a mobile treatment device), or treatment of patients in situations where high-end ventilators may be unavailable (such as in crisis situations, in temporary hospitals, in rural environments, in military settings, or in developing countries).

Further, it will be appreciated that exemplary embodiments of the software-defined ventilator platform described herein may not only be used to treat patients infected with COVID, but also patients having other diseases or conditions for which breath support may be needed. Additionally, for diseases such as COVID where early treatment and oxygen breath support are known to lead to improved outcomes, the availability of a low-cost and flexibly configurable breath support device in accordance with the software-defined ventilator platform is highly advantageous, allowing for early symptomatic patients to receive oxygen support. The software-defined ventilator platform may further provide network connectivity to enable remote monitoring and periodic or continuous assessment of the patient by health providers and/or a health provider system.

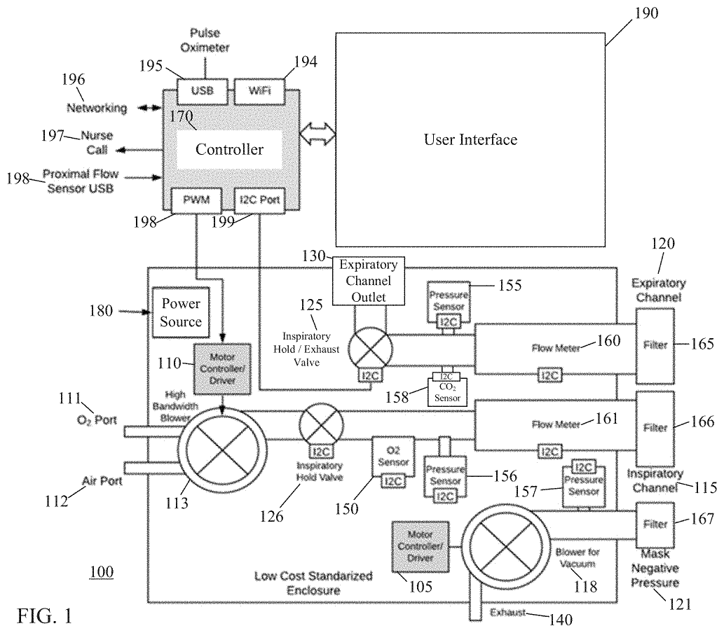

FIG. 1 is a block diagram 100 depicting a software-defined ventilator system according to an exemplary embodiment of the present application. The software-defined ventilator system includes a pneumatic system and a control system in communication therewith. The pneumatic system generates airflow, provides oxygen and air mixing, measures and senses airflow, and controls percentage of carbon dioxide and oxygen in the airflow. The control system interacts with sensors, actuators, and controllers/drivers of the pneumatic system to obtain data from the pneumatic system and to control the pneumatic system.

To provide for control over the amount of oxygen provided to the patient and the breath pressure for the patient, the control system may interact with the pneumatic system by obtaining data from a plurality of sensors disposed in the pneumatic system and by controlling various components of the pneumatic system, including motor controllers/drivers and valves. This enables a dynamically-configured and pressurized airflow to be provided to the patient, and the control system may utilize real-time feedback from the pneumatic system, which may include, for example, pressure, flow volume, oxygen level, carbon dioxide level, and other parameters being measured (e.g., at hundreds to thousands of times per second). This information may be used by healthcare providers to analyze a patient's lung function as it changes or progresses over time and/or by the system to adjust a treatment regimen on the fly.

In an exemplary embodiment, the pneumatic system includes two main pathways—i.e., an inspiratory channel 115 (for controlled inflow of air/oxygen to a patient's lungs) and an expiratory channel 120 (for controlled outflow of exhaled air). The two channels are connected to the patient via corresponding tubing and an apparatus such as a face mask or a nasal mask for providing non-invasive breath support (alternatively, other apparatuses may be used, such as a helmet for non-invasive breath support, or the tubing may go into a patient's airway for invasive breath support). In certain exemplary embodiments (e.g., when a face mask is used for non-invasive breath support), the pneumatic system may optionally further include a negative pressure channel 121 (which is another outflow channel) for ensuring a tight mask seal and for mitigating or avoiding the spread of contagions from the patient.

The inspiratory channel 115 of the pneumatic system, which is used to provide oxygen or a mixture of oxygen/air to the patient and/or to provide breath pressure for the patient, may include a blower 113 (e.g., a high bandwidth blower having a response time of less than 0.05 seconds), an inspiratory hold valve 126, an oxygen sensor 150, a pressure sensor 156, a flow meter 161, and a filter 166 (e.g., a biofilter which filters out viral or bacterial particles). The expiratory channel 120 of the pneumatic system, which is used to expel exhalations from the patient as well as to control the duration and/or rate of exhalation, may include an outlet 130, an inspiratory hold/exhaust valve 125, a pressure sensor 155, a carbon dioxide sensor 158, a flow meter 160, and a filter 165. As discussed above, the sensors, valves and blower interact with the control system of the software-defined ventilator system for control over the amount of oxygen provided to the patient and the breath pressure provided for the patient.

The blower 113 has an oxygen input 111 and an air input 112 and is controlled/driven by a motor controller/driver 110 of the pneumatic system which is in communication with the control system. The motor controller/driver 110 may be a brushless 3-phase motor controller/driver, which provides an appropriate current, voltage and frequency response to drive the blower 113. It will be appreciated that a brushless 3-phase motor controller may provide significant headroom for a decade-long service lifetime. In an exemplary implementation, the response time of the combination of the motor controller/driver 110 and the high bandwidth blower 113 may be under two thousandths of a second, and the combination is able to deliver pressure levels well over what would be needed a patient suffering from acute respiratory distress syndrome (ARDS) or from severe cases of COVID. Thus, this subsystem of the software-defined ventilator platform according to exemplary embodiments of the present application is able to deliver a level of pressure and response matching that of high-end, high-cost conventional ventilators.

The blower 113 is connected to an oxygen source, such as an oxygen tank, via an oxygen port 111. The high bandwidth blower 113 is further connected to an air source, such as the environment, via an air port 112. In an exemplary embodiment, the flow of oxygen to the blower 113 may be manually controlled via a manual valve connected to the oxygen source. In another exemplary embodiment, the flow of oxygen to the blower 113 may be electronically controlled by the control system via an electronic valve connected to the oxygen source (e.g., based on user input to the user interface 190 in response to oxygen level measurements displayed on the user interface 190 and/or using feedback-based control from controller 170). In another exemplary embodiment, the oxygen port 111 and the air port 112 may be combined into a single port which is connected to a blender, and the blender provides a customizable mix of oxygen and air to the blower 113 via the single port. The blender may be controlled manually (e.g., using a knob that goes from 0-100% oxygen) or may be controlled electronically via the control system. In some exemplary embodiments, the flow of oxygen is dynamically adjusted by the controller 170 based on real-time oxygen measurements from the oxygen sensor 150 or from a pulse oximeter (e.g., if the controller determines that the patient is not getting enough oxygen based on sensor readings, the controller may automatically increase the oxygen percentage being supplied to the patient). A healthcare provider may also utilize the user interface 190 to view oxygen measurements and, based thereon, input an oxygen setting or a desired treatment regimen via the user interface 190, thereby causing the controller 170 to control the pneumatic system accordingly to provide an appropriate amount of oxygen to a patient.

Providing control over the amount of oxygen being supplied to the patient is advantageous, for example, for treatment of COVID patients, who typically need a relatively high percentage of oxygen (e.g., a mixture of air/oxygen in a range of 50% to 100% oxygen) pushed into their lungs. Unlike conventional ventilators which utilize a complex mixing system for regulation of oxygen input, the software-defined ventilator system of FIG. 1 is able to provide a regulated flow of oxygen in a controlled manner without needing a complex mixing system.

The blower 113 is also able to provide a controllable amount of breath pressure for patients. On an inhale, the inspiratory hold/exhaust valve 125 on the expiratory channel is closed, and the blower 113 is controlled by the motor controller/driver 110 (based on signals from the controller 170) to provide a configurable amount of pressure and flow towards the patient's lungs. This is advantageous, for example, for treatment of ARDS patients to provide patients with an appropriate amount of assistance with respect to inflating their lungs. This is also advantageous, for example, for treatment of COVID patients who need oxygen support, as it allows for better inflating of a patient's lungs and for keeping them inflated for a longer period of time to increase oxygen uptake.

The blower 113 may also be controlled during an exhale (while the inspiratory hold/exhaust valve 125 is open) to provide a desired amount of positive expiratory end pressure (PEEP). Control of the PEEP may be based on feedback from the pressure sensor 115 and/or the pressure sensor 156. A PEEP value may be preset, for example, to be in a range from 5 to 25 cm H₂O. For COVID patients, a PEEP value of about 25 cm H₂O may be set.

The blower 113 and the other components of the inspiratory channel 115 work together with components of the expiratory channel 120 to provide a wide range of breath support functions. Information from the various oxygen sensors, pressure sensors, carbon dioxide sensors, and flow meters in the pneumatic system and/or from other sensors (such as a pulse oximeter and/or a proximal flow sensor) may be utilized in a variety of different ways to closely monitor a patient's breathing and to dynamically tailor a treatment regimen for the patient (either automatically by the system or with user input from a user such as a healthcare provider). Different modes of operation of the software-defined ventilator system may use and/or output different respective combinations of sensor feedback.

In certain exemplary implementations, the blower 113 may be similar to the type of blowers used in continuous positive airway pressure (CPAP) machines or bilevel positive airway pressure (BiPAP) machines, which are able to provide adequate headroom to achieve at least 50 cm H₂O maximum pressure.

The pneumatic system may also provide an “inspiratory hold” function using the inspiratory hold/exhaust valve 125 and the inspiratory hold valve 126. An “inspiratory hold” function provides the patients with a breath, and then closes both valves 125, 126 to prevent exhalation. While the inspiratory hold valves 125, 126 are closed, the control system measures, using pressure sensor 155 and/or pressure sensor 156, a plateau pressure in a patient's lungs. This provides information as to how stiff the patient's lungs are, and a healthcare provider may use this information to adjust a treatment regimen accordingly. In an exemplary embodiment, the control system may also automatically adjust the treatment regimen based on the plateau pressure measurement.

The pressure sensor 155 of the expiratory channel 120 and the pressure sensor 156 of the inspiratory channel 115 sense pressure in the respective channels. The oxygen sensor 150 of the inspiratory channel 115 senses an oxygen level of the breath being supplied through the inspiratory channel 115. The carbon dioxide sensor 158 of the expiratory channel 120 senses the carbon dioxide level of exhaled air in the expiratory channel 120. The flow meter 160 of the expiratory channel 120 and the flow meter 161 of the inspiratory channel 115 sense the rate of air flow in the respective channels.

As mentioned above, the pneumatic system of FIG. 1 may further include a negative pressure channel 121. The negative pressure channel may include a blower 118 (e.g., a vacuum blower) which is driven/controlled by motor controller/driver 105, as well as an exhaust 140 and a pressure sensor 157. Using readings from the pressure sensor 157, the control system can adjust the amount of power used to drive the blower 118 to maintain a small amount of negative pressure and create a seal for a mask worn by the patient. This in effect serves like a negative pressure room or a negative pressure hood environment which prevents virus particles exhaled by the patient from leaking out and contaminating the environment around the patient. Rather, the virus particles are caught in filters 165, 166 and 167, which provide biofiltration. Blower 118 may be of similar design as blower 113, but since it is used to maintain a gentle negative pressure, blower 118 does not need to be as powerful as blower 113.

Alternatively, instead of being attached to a mask worn by the patient, the negative pressure channel 121 may be attached to a secondary hood (e.g., a hard hood or a soft hood) disposed around the patient's head. The negative pressure is set to be strong enough to avoid leakage of viral particles outside of the hood but is gentle enough to avoid having the hood collapse over the patient's head.

It will be appreciated that the negative pressure channel 121 is operated independently relative to the expiratory channel 120 and the inspiratory channel 115. The negative pressure channel 121 is generally continuously operating during operation of the software-defined ventilator system to create a negative pressure space (e.g., within a patient's face mask or within a secondary hood around a patient's head).

It will be appreciated that in invasive use cases (i.e., where the inspiratory and expiratory channels are connected directly into a patient's airway instead of connected to a mask externally attached to the patient), aerosol release is less of a concern. As such, the negative pressure channel 121 and the components thereof may be omitted or may be unused in certain invasive use cases.

The pneumatic system may further include a power source disposed thereon, which may include, for example, a power input (e.g., 19V 3 A laptop charger) with power conditioning circuit and/or a battery pack.

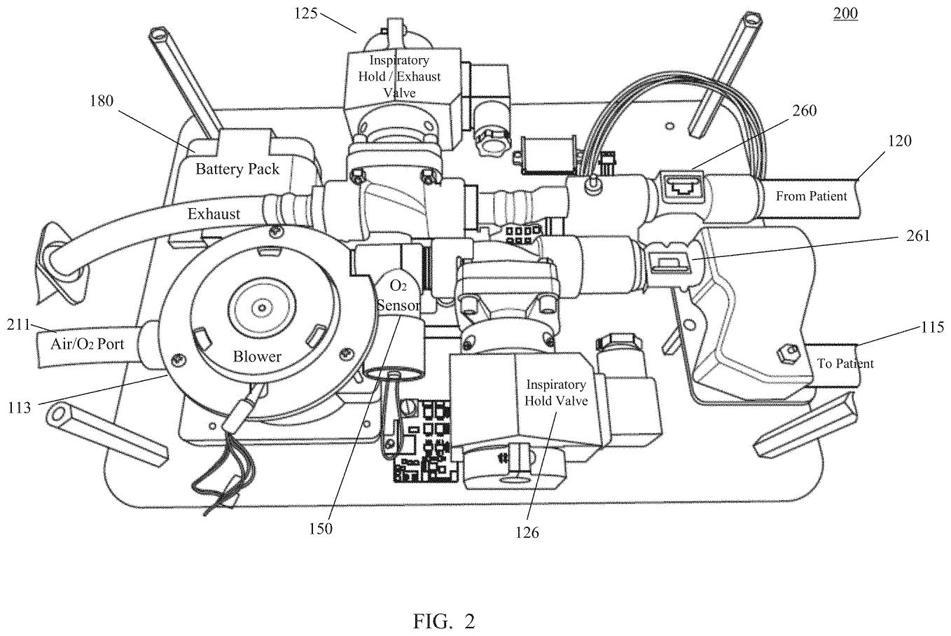

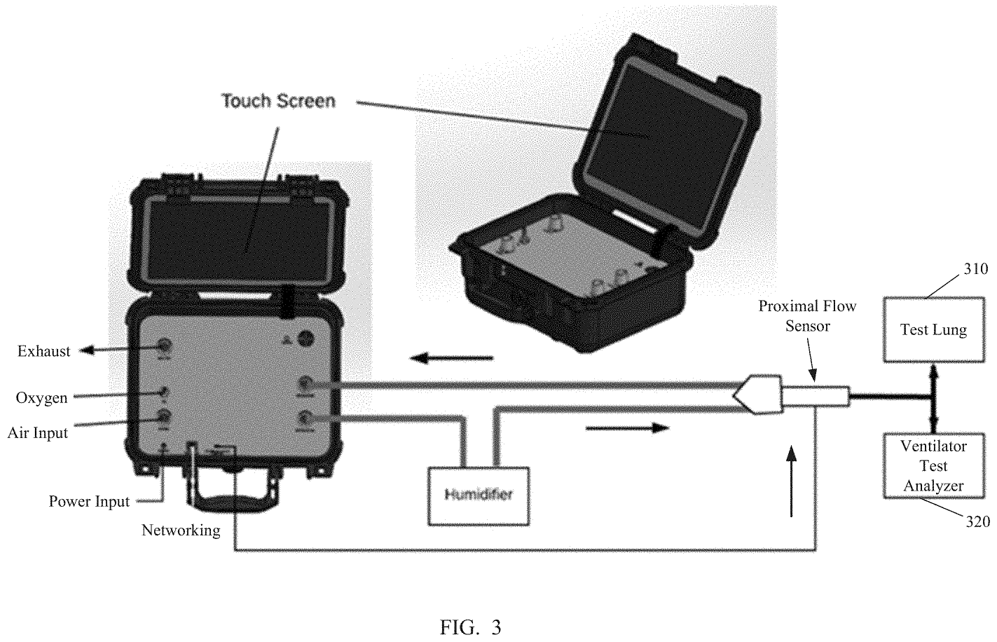

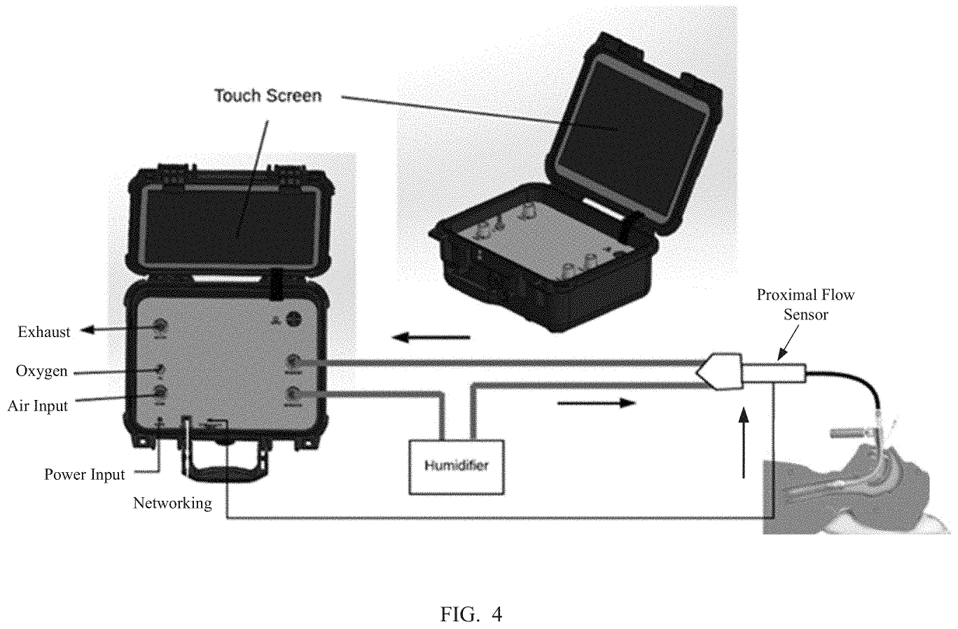

FIG. 2 is a schematic diagram 200 depicting a pneumatic system of a software-defined ventilator system according to an exemplary embodiment of the present application. As shown in FIG. 2, the pneumatic system has a modular design which allows the pneumatic system to be enclosed in a housing (e.g., in a plastic or metal structure). For example, a low cost standardized enclosure or case may be used to house and protect the pneumatic system (e.g., as depicted in FIGS. 3-4 below). The modular design also allows for the pneumatic system to be implemented in a variety of situations, such as in a portable software-defined ventilator package, in a multi-functional medical device, or in a fixed space, such as within an ambulance or a hospital room.

The pneumatic system of FIG. 2 is similar to the pneumatic system depicted within FIG. 1, except that it omits the negative pressure channel and includes some implementation differences. For example, instead of having separate air and oxygen ports as shown in FIG. 1, a single air and oxygen port 211 is input into blower 113 of FIG. 2. Additionally, instead of having separate pressure and flow sensors in the inspiratory channel 115 and the expiratory channel 120, a combined flow/pressure sensing device 261 is used in the inspiratory channel 115 and a combined flow/pressure sensing device 260 is used in the expiratory channel 120.

Referring back to FIG. 1, the control system in communication with the above-discussed examples of the pneumatic system may include a controller 170 and a user interface 190. The user interface 190 may include, for example, a touchscreen display. The user interface 190 may further include, for example, one or more mechanical components (such as buttons or knobs) disposed on an enclosure for the pneumatic system and/or the control system. The user interface 190 may be implemented based on any of a wide array of human interface options readily available for controllers and micro-computers, for example, as used in computers, laptops, tablets, and smartphones.

The controller 170 may be, for example, a programmable micro-computer such as an Arduino or Raspberry Pi device. The controller 170 may have substantial processing power, may be able to run standard operating systems, and may have direct interfaces for communication with components of the pneumatic system, as well as communication with other external systems (such as a nurse call system, an external network, or peripheral devices).

The controller 170 may include, for example, an I2C port 199 for communication with the pressure sensors, flow meters, and valves of the pneumatic system. An I2C communication line is illustrated in FIG. 1 between I2C port 199 and inspiratory hold valve 125, and it will be appreciated that other I2C communication lines may also be implemented between the I2C port 199 and the various components labeled with “I2C” in FIG. 1. Thus, unlike conventional ventilators in which analog components are deployed and communicate with embedded hardware components in a costly and complex manner, exemplary embodiments of the present application are able to utilize digital components which provide digital data and are amenable to digital control for greater flexibility and lower cost. In certain exemplary embodiments, intra-device communications between the components illustrated in FIG. 1 may utilize I2C, and communications with other devices or components (e.g., pulse oximeter, proximal flow sensor, etc.) outside of the main device may be performed via USB (e.g., I2C over USB), WiFi, or near-field communications (e.g., Bluetooth). Other forms of wired and wireless communications may also be utilized for communications between components.

The controller 170 may further include a pulse width modulation (PWM) output 198 for communication with and control of one or more motor controllers/drivers. A PWM communication line is illustrated in FIG. 1 between PWM port 198 and motor controller/driver 110, and it will be appreciated that another PWM communication line may be implemented between PWM port 198 and motor controller/driver 105.

The controller 170 may further include a Universal Serial Bus (USB) port 195 for communication with a pulse oximeter attached to the patient. The pulse oximeter may be used to measure a patient's blood oxygen level. The USB port 195 (or a plurality of USB ports 195) may also be used for other types of devices, such as attachment to electrocardiogram (ECG) devices, body temperature sensors, blood pressure monitors and other types of monitoring devices, thereby providing for comprehensive monitoring of a patient's status, e.g., to provide relevant information for an intensive care unit (ICU) environment.

The controller 170 may further include a proximal flow sensor USB connection 198 which provides measurements from a proximal flow sensor disposed relatively close to the patient (see, e.g., placement of proximal flow sensor in FIGS. 3-4). As depicted in FIGS. 3-4, generally, between a ventilator and a patient, there is a tube network (long or short) and a humidifier chamber, which is often referred to as “dead air space.” The proximal flow sensor may provide real-time volume and/or flow rate measurements with regard to the oxygen/air mixture being pushed into a patient's lungs and with regard to an exhalation by the patient. The information provided by the proximal flow sensor may be used to supply air to a patient in prescribed intervals, and may further be used for diagnostic purposes to guide clinicians in making the optimal treatment decisions. Precise breath control is particularly advantageous for pediatric patients who have relatively small lung capacity, and locating the proximal flow sensor as close as possible to the patient's airway is advantageous to provide more precise flow measurements based upon which the precise control can be provided by the software-defined ventilator system. Thus, the proximal flow sensor may be located as close as possible to a patient's airway (e.g., within a tubing circuit at a location proximate to a face mask to which the tubing circuit is attached).

The controller 170 may further include a nurse call connection 197. The nurse call connection 197 may be used, for example, to notify emergency medical services (EMS) and/or trigger an alarm in case a patient's condition deteriorates. For example, this may be manually triggered by the patient in the event of an emergency and/or automatically triggered by the system upon certain conditions being met (e.g., based upon the patient's oxygen level falling below a certain level or other indicators of a patient's worsening condition). The nurse call connection 197 may thus facilitate the patient getting medical assistance in a timely manner in the event of an emergency.

The controller 170 may further include wireless network capabilities (e.g., a WiFi interface 194 and/or a Bluetooth interface) and/or other networking connections 196, to provide additional functionality to the user through the control system. Further, the user interface 190 may include a video camera device, which together with a networking connection 196 may allow for health care providers to evaluate, diagnose, and/or treat patients through telemedicine. One or more networking connections 196 may also be used to provide patient data to respiratory therapists or other healthcare providers for regularly monitoring the conditions of patients. The wireless network capabilities also enable the software-defined ventilator system to be remotely controlled, which is particularly advantageous in situations where healthcare providers may not be able to be on-site to supervise treatment.

The controller 170 further includes a non-transitory memory which stores executable instructions which configure the controller 170 to communicate with and control the pneumatic system in order to implement a variety of treatment protocols and regimens. As such, the controller 170 may be flexibly programmed and/or updated, for example, via the WiFi interface 194, the USB interface 195, or other networking connections 196.

In view of the foregoing, it can be seen that the software-defined ventilator system provides a pneumatic system which may be thought of as a standardized pneumatic kernel, which is flexibly controllable by an adaptable software-based control system, and the control system in turn is flexibly attachable to a variety of external devices and systems. Thus, the software-defined ventilator system is able to provide a robust and flexible platform having a low-cost, standardized design which is useable for a variety of breath support treatment operations. As such, the drawbacks of conventional ventilators can be avoided with respect to requiring complex customized designs (via customized development libraries, operating systems, and support circuitry), complicated manufacturing, and costly, specialized hardware. For the software-defined ventilator platform according to exemplary embodiments of the present application, the development cycle can be significantly shortened relative to conventional ventilators, and because the software-defined ventilator platform utilizes standardized interfaces and a standardized software platform (in contrast to the specialized hardware/firmware/software used for conventional ventilators), it better facilitates cooperation for developers around the world to collaborate in developing updated treatment regimens in times of crisis. For example, an expandable software tree may be utilized to create opportunities for collaborators to add new features and graphical user interfaces (GUIs).

It will be appreciated that the arrangement of components in FIG. 1 is exemplary, and that other exemplary embodiments may include other arrangements of components. For example, additional sensors may be added such that both the inspiratory channel 115 and the expiratory channel 120 each have an oxygen sensor. Additionally, it will be appreciated that various exemplary embodiments of the ventilator system can be adapted to various deployment scenarios from in hospital use to developing world healthcare scenarios, to emergency and other field hospitals, to home use. In each of these deployment scenarios, adaptations of the ventilator system to the particulars of the deployment environment may be made, for example, with respect to powering, communications, type of enclosure (e.g., portable case, built-in enclosure), etc. Further, to provide medical care with communications and advanced services, a number of different sensor suites (including but limited to one or a combination of temperature, blood pressure, heart EKG, or blood oxygen sensors) may be built into the ventilator system and/or connected to the ventilator system via, for example, USB or Bluetooth connections.

FIG. 3 is a schematic/block diagram depicting a test environment for a software-defined ventilator system according to an exemplary embodiment of the present application. In the example shown in FIG. 3, an exemplary embodiment of the control system and the pneumatic system depicted in FIG. 1 is implemented in a portable case (e.g., a Pelican case or a modified version of a Pelican case). This may be thought of an “ICU in a suitcase” example which is able to provide ventilator functionality for patients that would otherwise typically only be available in an ICU setting. The “ICU in a suitcase” design thus allows for advanced ventilator functionality to be accessible to patients in their homes, in public spaces, and in vehicles. This design also allows for stockpiling of ventilators to be held in reserve in the event of a crisis such as a pandemic, and easy deployment of such ventilators in overloaded hospitals, in temporary hospitals, in rural environments, in military contexts, and/or in developing countries.

The test environment includes the modular pneumatic system disposed in the case, with various inputs and outputs of the pneumatic system being accessible via ports disposed on the case (e.g., an exhaust channel output port, an exhaust channel input port, oxygen and air input ports for an inspiratory channel, an output port for the inspiratory channel connected to a humidifier, a power input, a connection to a proximal flow sensor, and a networking port). Further, a touchscreen (e.g., an 11-inch touchscreen connected to a Raspberry Pi micro-computer) may be embedded in the lid of the case.

In some exemplary implementations, High Definition Multimedia Interface (HDMI) and USB ports may be provided on the case for connection to larger screens (for example, in relatively more permanent installations).

In some exemplary implementations, Ethernet networking may be provided via the networking port for network integration of the software-defined ventilator system and for providing upgrades to the software-defined ventilator system.

In some exemplary implementations, a model-view-presenter (MVP) GUI design pattern may be utilized.

In some exemplary implementations, a proportional-integral-derivative (PID) feedback control mechanism may be implemented in the controller.

The test environment depicted in FIG. 3 was used to test the operation of an exemplary embodiment of the software-defined ventilator system of FIG. 1 on a test lung apparatus 310 (e.g., a Michigan Instruments test lung). The performance of the system was analyzed by a ventilator test analyzer 320 (e.g., a Fluke ventilator test analyzer). During the testing, the blower was connected to an RC motor controller and a PWM output of a Raspberry Pi micro-computer within the case, and short pulses at anticipated breath rates were simulated. Based on these simulations, it was demonstrated that the software-defined ventilator system of FIG. 1 is able to satisfy the operating requirements for a wide range of applications. It will also be appreciated that the test environment depicted in FIG. 3 and the ventilator test analyzer 320 may be utilized to calibrate sensors of the pneumatic system of FIG. 1.

FIG. 4 is a schematic/block diagram depicting a treatment environment for a software-defined ventilator system according to an exemplary embodiment of the present application. FIG. 4 is similar to FIG. 3, except that the software-defined ventilator system is depicted as being connected to a patient in FIG. 4 (instead of being depicted as connected to a test lung apparatus 310 and a ventilator test analyzer 320). In the example shown in FIG. 4, an invasive breath support operating mode is depicted. In this mode, a patient's breathing effort is minimized with assistance from an artificial airway (e.g., via an endotracheal tube or a tracheostomy tube) into and out of the patient's lungs. In other exemplary embodiments, it will be appreciated that the treatment environment may include the use of the software-defined ventilator system for a non-invasive breath support operating mode, where breath support is provided via a face mask, a nasal mask, or a helmet.

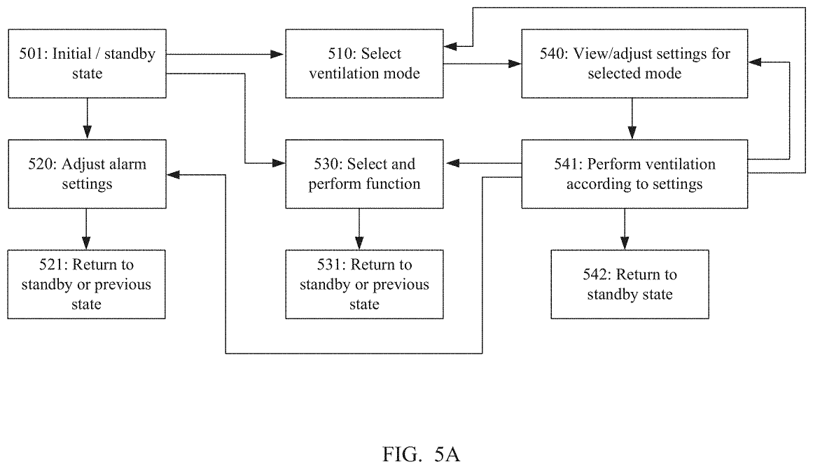

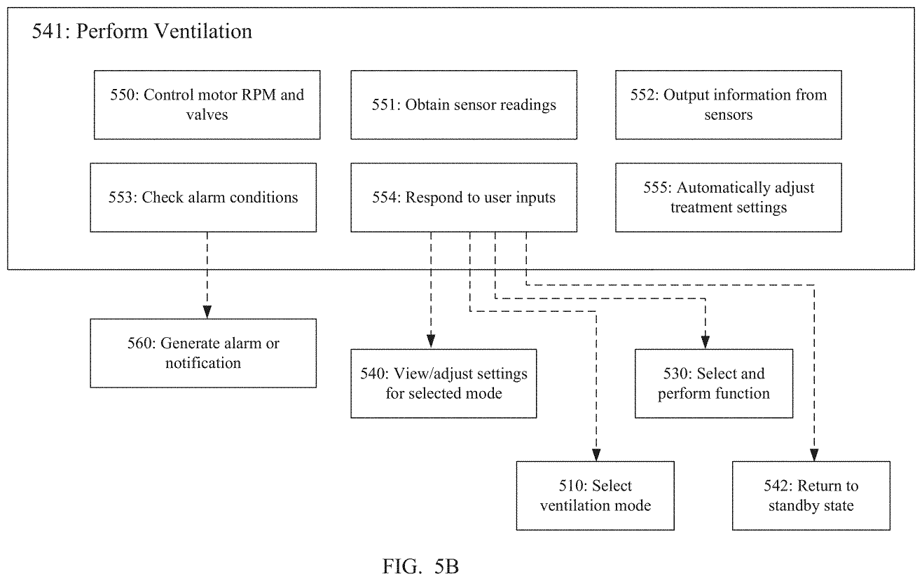

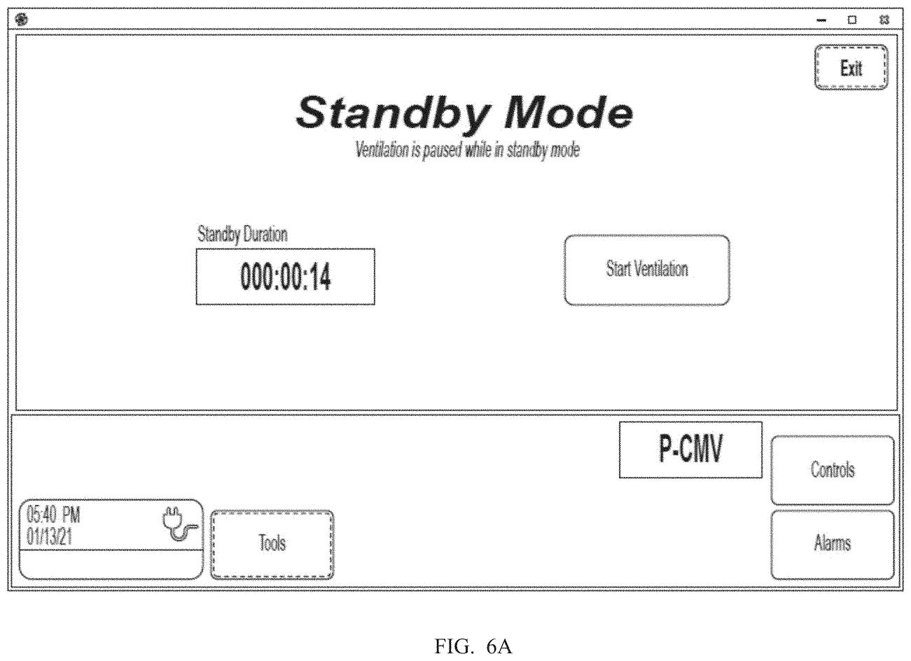

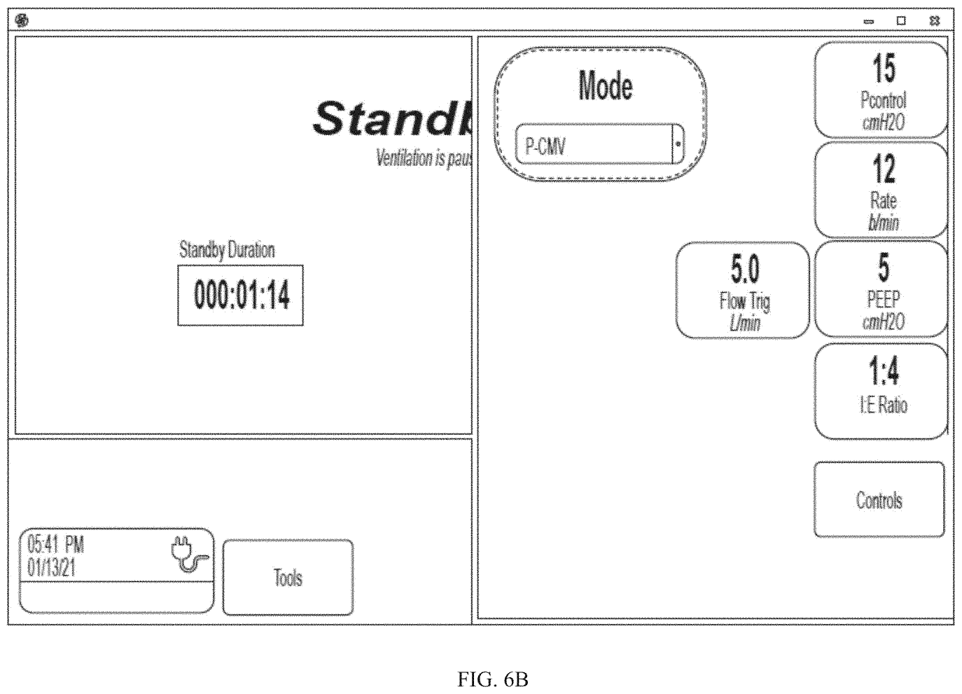

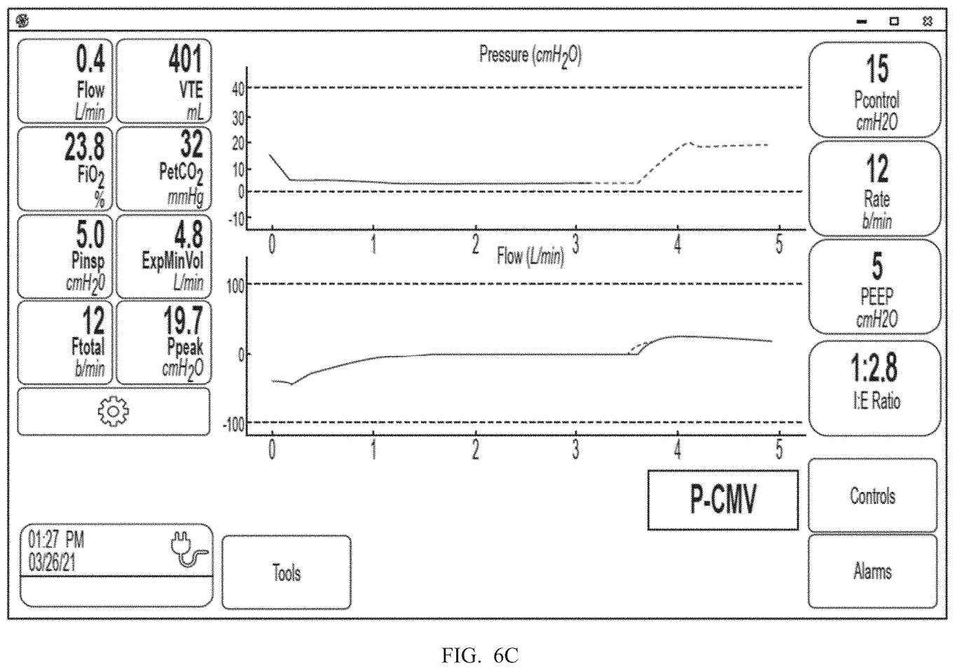

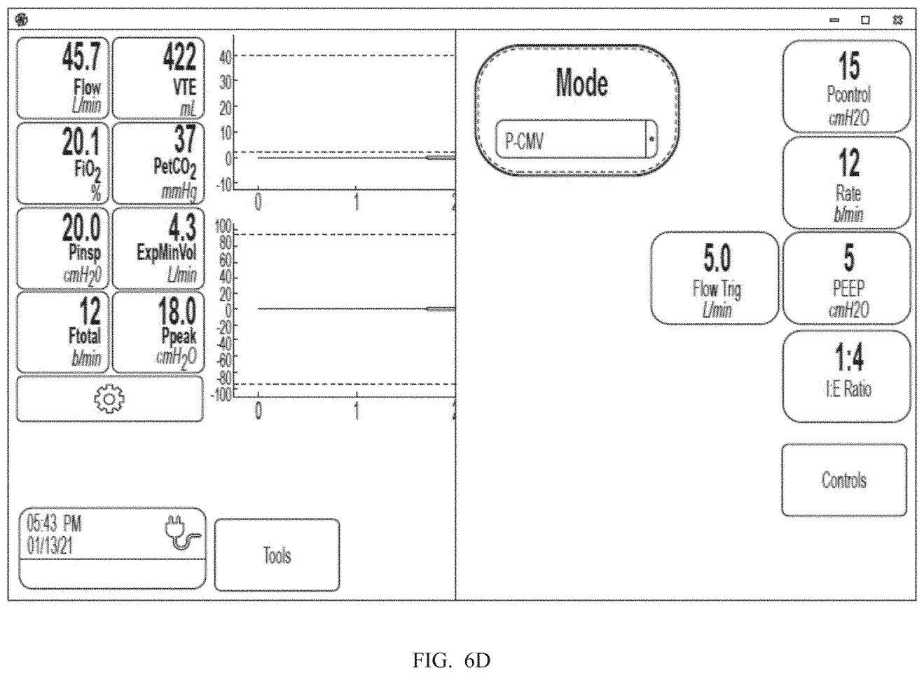

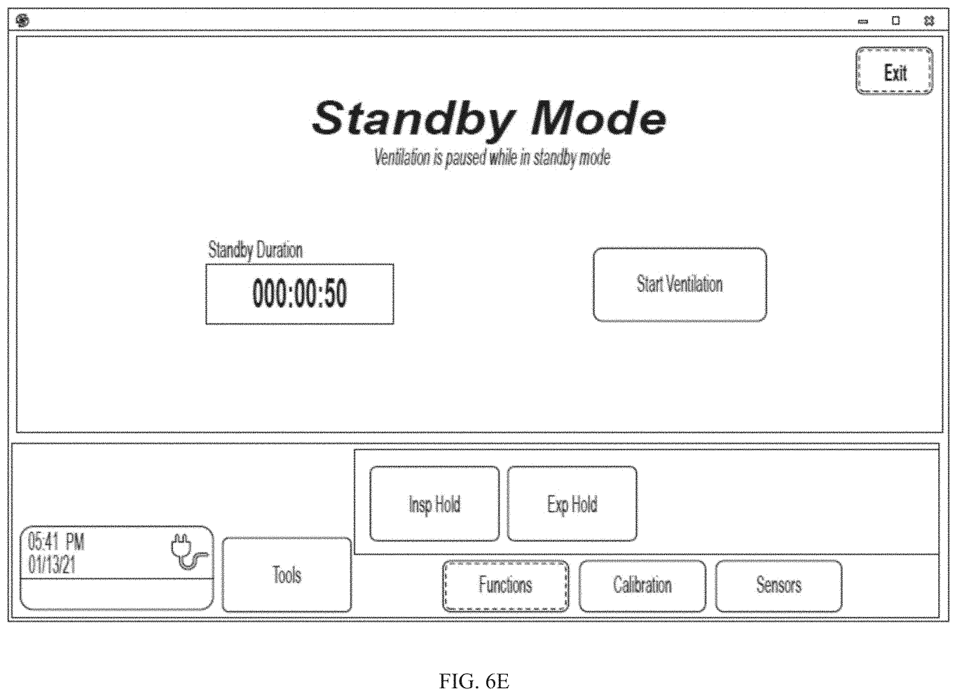

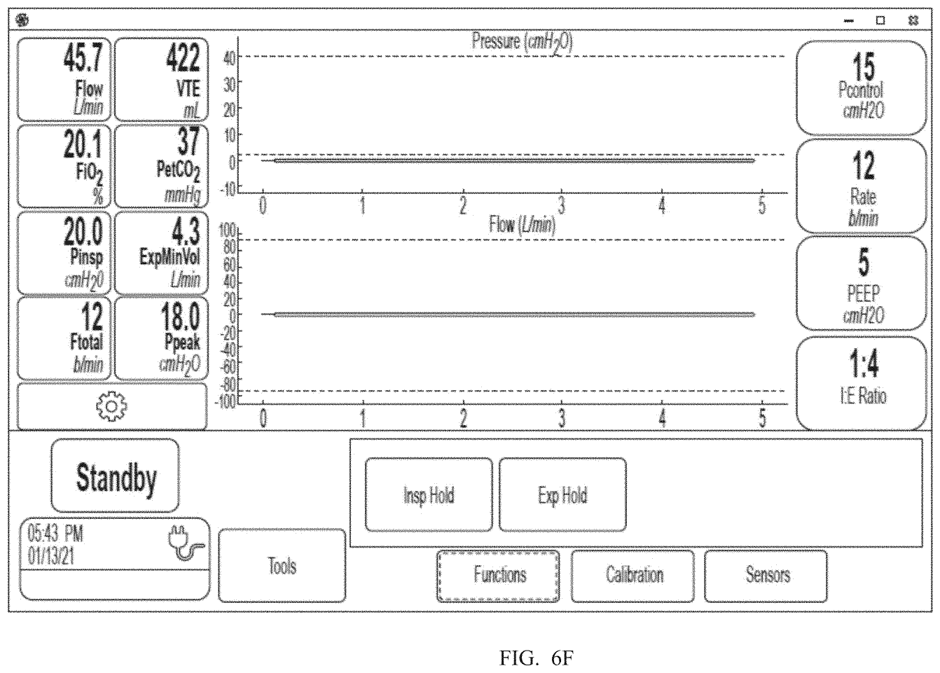

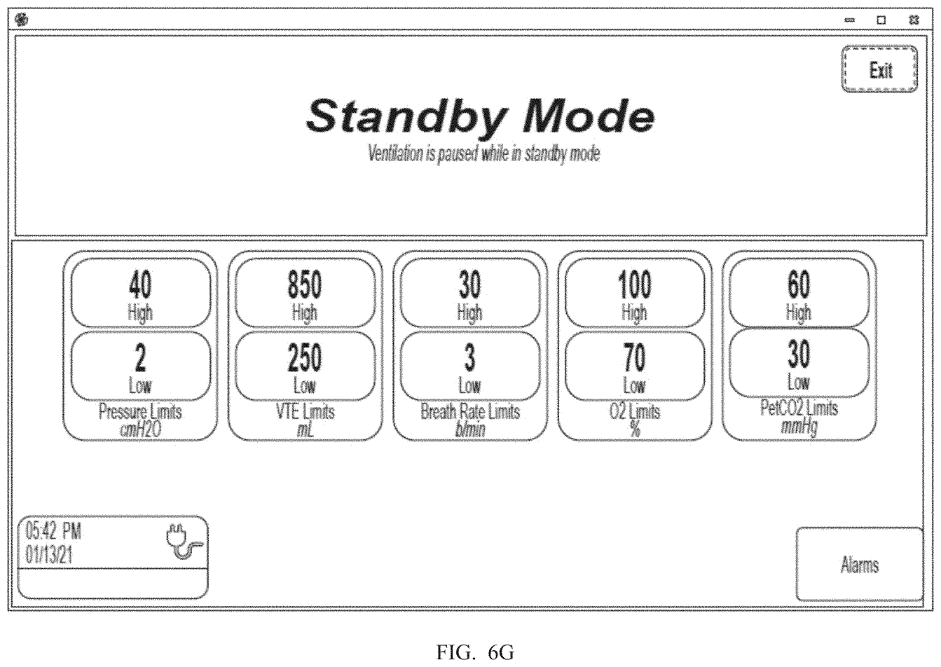

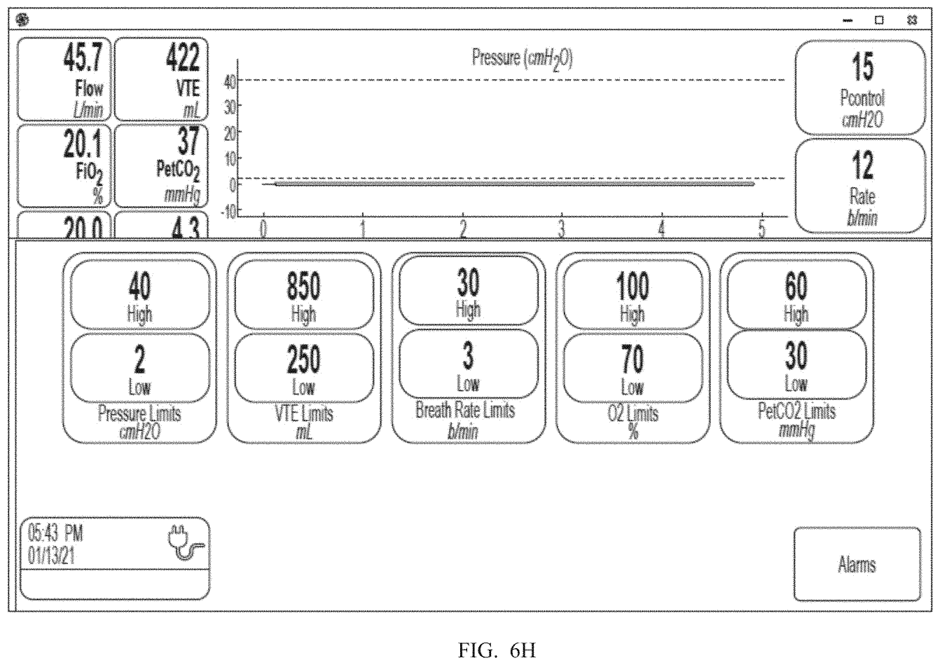

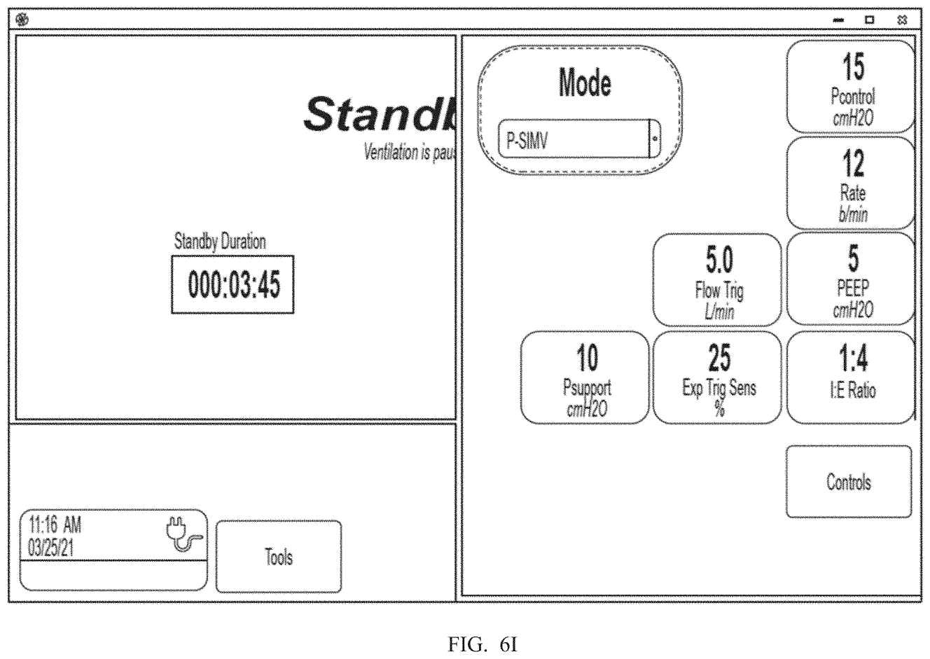

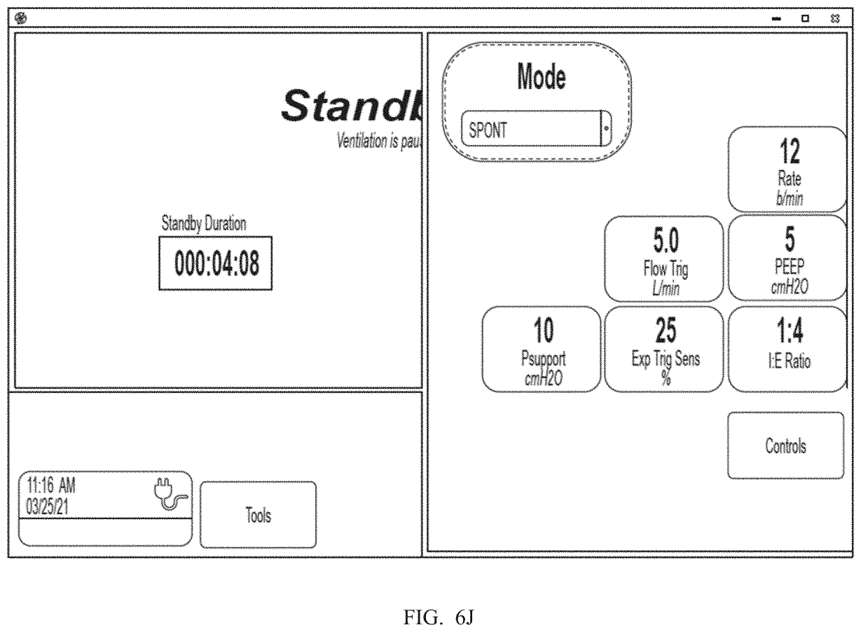

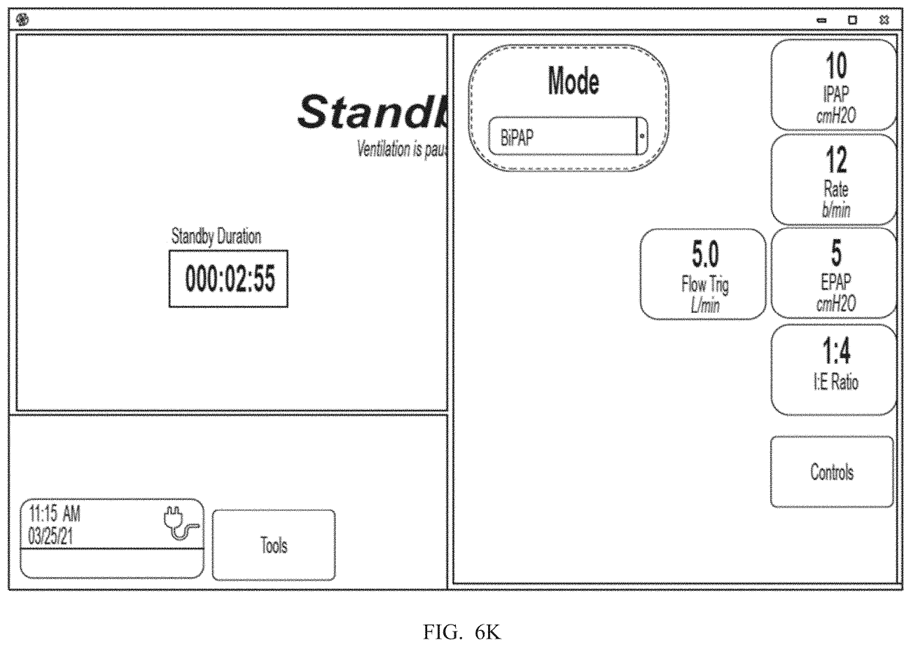

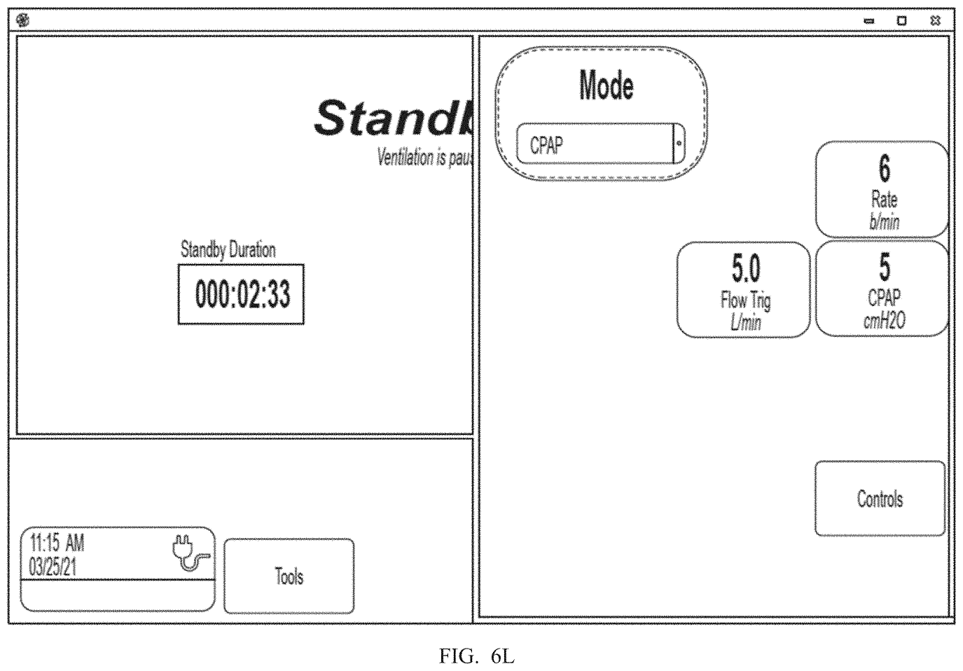

FIGS. 5A-5B are exemplary flowcharts depicting a process for operating a software-defined ventilator system according to an exemplary embodiment of the present application, and FIGS. 6A-6L are exemplary illustrations depicting user interfaces of a software-defined ventilator system according to an exemplary embodiment of the present application. The process depicted in FIGS. 5A-5B may be implemented, for example, on controller 170 of the software-defined ventilator system depicted in FIG. 1, and the graphical user interfaces (GUIs) depicted in FIGS. 6A-6L may be displayed and interacted with via user interface 190 of the software-defined ventilator system depicted in FIG. 1, wherein the user interface 190 includes a touchscreen.

In FIG. 5A, stage 501 corresponds to an initial or standby state of the ventilator system. For example, when the ventilator is turned on or reset, it may enter this state. In another example, when a treatment is completed or paused, the ventilator may also enter this state. FIG. 6A depicts an exemplary GUI corresponding to the initial or standby state. As can be seen from FIG. 6A, the GUI may provide options to a user with respect to proceeding to a ventilation treatment, adjusting alarm settings, or performing other functions.

If a user wishes to proceed with activating a ventilation treatment, the controller may receive a user input selecting a ventilation mode at stage 510, and the controller may provide the user with the ability to view and adjust settings for the selected ventilation mode at stage 540. FIG. 6B depicts an exemplary GUI on which a ventilation mode and setting adjustment overlay is displayed over the standby state GUI. Using this interface, a user can select a ventilation mode from a drop-down menu (e.g., P-CMV is shown as the currently selected mode in FIG. 6B), and can adjust various configurable parameters (e.g., Pcontrol, Rate, Flow Trig, PEEP, and I:E Ratio as shown in FIG. 6B) corresponding to the currently selected mode. FIGS. 6I-6L depict additional examples of configurable parameters with respect to the P-STMV mode, the SPONT mode, the BiPAP mode, and the CPAP mode, respectively.

At stage 541, a ventilation treatment is carried out by the ventilator system according to the selected mode and the selected settings. The ventilation treatment may include a series of repeating “breath loops” (also referred to herein as “respiratory cycles”) which each include an inhalation portion and an exhalation portion. The ventilator system may provide a respective manner of assistance and control for the inhalation portion and/or exhalation portion of each breath cycle depending on the selected mode and the settings of the selected mode.

FIG. 6C depicts an exemplary GUI which may be output by the controller on the user interface while the treatment is ongoing. The output GUI may include various information regarding the treatment and the patient, including for example, Flow, VTE, FiO₂, PetCO₂, Pinsp, ExpMinVol, Ftotal, Ppeak, a pressure over time plot, a flow over time plot, the current mode (P-CMV), and current settings (Pcontrol, Rate, PEEP, I:E Ratio). The pressure over time plot may provide information regarding the set pressure profile over time (shown with the dotted line) and a measured pressure profile over time (shown with the solid line, which is updated as time passes and corresponding data is gathered). Similarly, the flow over time plot may provide information regarding the set flow profile over time (shown with the dotted line) and a measured flow profile over time (shown with the solid line, which is updated as time passes and corresponding data is gathered).

While the ventilation treatment is ongoing at stage 541, the controller may receive further user input to select a different mode of operation for the ventilator system (in which case the process proceeds back to stage 510) or to adjust settings of the current mode of operation (in which case the process proceeds back to stage 540). FIG. 6D depicts an exemplary GUI which may be output by the controller on the user interface, and which provides the user with options to adjust the mode of operation and/or the settings for the selected mode of operation in a manner where the adjustment menu is overlaid over the treatment GUI of FIG. 6C.

When a treatment is over or when the controller receives a user input pausing or stopping the treatment, the controller may return the ventilator system to the initial/standby state at stage 542.

The ventilator system may also provide for other functions in addition to various modes of ventilation treatment. At stage 530, the controller may receive a user input selecting one of these other functions and cause the corresponding function to be performed. In the example depicted in FIG. 5A, the process may flow to stage 530 from the initial/standby state at stage 501, or the process may flow to stage 530 during a ventilation treatment at stage 541. FIG. 6E depicts an exemplary GUI which may be output by the controller on the user interface when a function menu is accessed from the standby mode GUI of FIG. 6A, and FIG. 6F depicts an exemplary GUI which may be output by the controller on the user interface when a function menu is accessed from the treatment GUI of FIG. 6C.

Different functions may be available depending on whether stage 530 is executed from stage 501 or from stage 541. For example, the other functions that may be performed by the ventilator system may include an inspiratory hold function, an expiratory hold function (similar to the inspiratory hold function for determining a plateau pressure, but for allowing time for pressure to equalize between the different parts of the respiratory circuit), a calibration function, or a function to view sensor information. The calibration function may be disabled, for example, if the function menu is activated while there is an ongoing ventilation treatment at stage 541.

The ventilator system may also provide for various alarms or notifications to be generated in response to detecting certain conditions being met. For example, high and low limits may be set for a plurality of parameters, including pressure, VTE, breath rate, O₂ and PetCO₂. Alarm limits utilized by the ventilator system may be adjusted at stage 520, for example, based on user input. FIG. 6G depicts an exemplary GUI which may be output by the controller on the user interface when an alarm adjustment menu is accessed from the standby mode GUI of FIG. 6A, and FIG. 6H depicts an exemplary GUI which may be output by the controller on the user interface when an alarm adjustment menu is accessed from the treatment GUI of FIG. 6C. In certain exemplary embodiments, each mode has a respective set of default alarm limits, and alarm limits may be adjusted by a user specifically for a respective mode without affecting alarm limits in other modes.

In FIG. 5B, operations that may be performed in connection with stage 541 are depicted in more detail. In particular, in this example, during the ventilation treatment provided by the ventilator system, the controller may control motor revolutions per minute (RPMs) and valves in accordance with settings of a current treatment regimen (stage 550), obtain sensor readings (stage 551), output information from the sensors (stage 552), check alarm conditions (stage 553), respond to user inputs (stage 554), and automatically adjust treatment settings (stage 555).

At stage 550, in order to control motor RPMs, the controller may communicate with a motor controller/driver of a pneumatic system (e.g., as depicted in FIG. 1), and utilize sensor values and/or stored algorithms to appropriately provide control signals to the motor controller/driver in accordance with the treatment settings. The controller may also appropriately control valves of the pneumatic system to accomplish respective operations (such as closing an exhaust valve during an assisted inhalation and opening the exhaust valve for an exhalation).

At stage 551, the controller may communicate with pressure sensors, flow sensors, oxygen sensor(s), carbon dioxide sensor(s), a proximal flow sensor, a pulse oximeter, and other sensors or devices to obtain information relating to an ongoing treatment and/or to a patient. The information obtained from these sensors and/or devices may be used by the controller to control components of the pneumatic system at stage 550, to output information to a user (e.g., via a user interface or over a network) at stage 552, to check alarm conditions at stage 553, or to automatically adjust treatment settings at stage 555.

At stage 553, based on an alarm condition being checked and the controller determining that a monitored parameter falls below a lower alarm limit or exceeds an upper alarm limit, an alarm or notification may be generated at stage 560. The alarm or notification may be generated, for example, via the user interface of the ventilator system or may be communicated remotely to another computing device. The alarm or notification may also utilize a nurse call system to notify a healthcare provider of the alarm condition.

The controller may also respond to user inputs at stage 554 while a treatment is ongoing. For example, the user may provide user input which causes the controller to proceed to stage 540 for viewing/adjusting settings of a current treatment regimen, proceed to stage 510 for selecting a different ventilation mode, proceed to stage 530 for selecting and performing another function (such as an inspiratory hold function), or proceed to stage 542 by pausing or stopping the ventilation treatment and putting the ventilator system back into a standby state.

As discussed above, exemplary embodiments of the present application provide a flexible and low-cost software-defined ventilator platform which is able to perform a variety of treatments on a standardized pneumatic kernel. The following table provides just some illustrative examples of different operating modes that the software-defined ventilator system (including some exemplary lists of adjustable settings and exemplary sequences of operations). It will be appreciated that this list of modes is not intended to be limiting, and that other modes may also be provided in other exemplary embodiments.

TABLE 1
Exemplary Operating Modes

1. Continuous Positive Airway Pressure (CPAP) Mode
The system delivers a constant positive pressure into the airway during the entire respiratory
cycle.
Settings
CPAP—The pressure to deliver, [2, 28] cmH2O
Rate—Backup breath rate to ensure patient is breathing, [4, 33] breaths per minute
Flow Trigger—Trigger for patient breath detection; used for timing breath intervals, [1.0,
20.0] liters per minute
Oxygen—What percentage of oxygen in the air to deliver, [20, 100] percent.
Operation Sequence
1. Set motor to spin at calculated RPM to achieve CPAP setting pressure
2. Measure difference between pressure sensor and CPAP setting each time pressure setting is
read and modify RPM to correct difference, if appropriate
3. Each time flow sensor is read, activate flow trigger if the measured value is higher than or
equal to the flow trigger setting
4. When the flow trigger is activated, record patient breath occurring. Calculate breaths per
minute from this record and activate breaths per minute alarm if the value is out of alarm upper
or lower limits.
2. Biphasie Positive Airway Pressure (BiPAP) Mode
The system delivers time-cycled high and low positive pressure into the airway, i.e. low pressure
during exhalation, high pressure during inspiration
Settings
IPAP—The inspiratory pressure to deliver, [2, 28] cmH2O. This setting means breaths
are pressure controlled
EPAP—The expiratory pressure to deliver, [2, 28] cmH2O
Rate—Set breath rate, used with I:E ratio to time cycle the two pressure values, [4, 33]
breaths per minute
I:E Ratio—Inspiratory to Expiratory ratio, used with breath rate to time cycle breath
delivery, [1:8, 8:1]
Flow Trigger—Trigger for patient breath detection; used for timing breath intervals, [1.0,
20.0] liters per minute
Oxygen—What percentage of oxygen in the air to deliver, [20, 100] percent.
Operation Sequence
1. Set motor to spin at calculated RPM to achieve EPAP setting pressure in the airway
2. Use I:E ratio and breath rate to determine time of inspiration and time of expiration
3. Set motor to spin at calculated RPM to achieve IPAP setting pressure in the airway and close
expiratory valve
4. After time of inspiration passes (e.g., a number of seconds), measure difference between peak
breath pressure and IPAP setting to change calculated RPM to achieve IPAP
5. Set motor to spin at calculated RPM to achieve EPAP setting pressure in airway and open
expiratory valve
6. Each time the flow sensor is read, activate the flow trigger if the measured value is higher than
or equal to flow trigger setting
7. When the flow trigger is activated, record patient breath occurring. Calculate breaths per
minute from this record and activate breaths per minute alarm if the value is out of alarm upper
or lower limits
3. Pressure Controlled inverse Ratio Ventilation (PC-IRV) Mode
Similar to Pressure Controlled CMV with LE ratio flipped—i.e., the majority of time is spent at
high inspiratory pressure.
Settings
PEEP—Constant pressure to maintain while not in inspiration, [2, 28] cmH2O
Pcontrol—High pressure value to deliver during inspiration, [2, 28] cmH2O. This setting
means breaths are pressure controlled
Rate—Set breath rate, used with I:E ratio to time cycle breaths, [4, 33] breaths per minute
E:I Ratio—Expiratory to inspiratory ratio, used with breath rate to time cycle breath
delivery, [1:8, 8:1]
Flow Trigger—Trigger for patient breath detection; used for timing breath intervals and
timing delivery of Psupport pressure, [1:8, 8:1] liters per minute
Oxygen—What percentage of oxygen in the air to deliver, [20, 100] percent.
4. Airway Pressure Release Ventilation (APRV) Mode
The system delivers time cycled high and low pressure with longer time spent at high pressure.
Allows spontaneous patient breaths
Settings
Phigh—The pressure to deliver for inspiratory time, [2, 28] cmH2O. This setting means
breaths are pressure controlled
Plow—The pressure to deliver for expiratory time, [0, 28] cmH2O
Rate—Set breath rate, used with I:E ratio to time cycle breaths, [4, 33] breaths per minute
I:E Ratio—Inspiratory to Expiratory ratio, used with breath rate to time cycle breath
delivery, [1:8, 8:1]
Flow Trigger—Trigger for patient breath detection; used for timing breath intervals and
recording spontaneous breaths, [1.0, 20.0] liters per minute
Oxygen—What percentage of oxygen in the air to deliver, [20, 100] percent.
5. Spontaneous (SPONT) Mode
Variation of Pressure Controlled SIMV with no guaranteed breaths—i.e., the patient initiates all
breathing activity and all breaths are supported breaths
Settings
PEEP—Constant pressure to maintain while not in inspiration, [2, 28] cmH2O
Psupport—Pressure to deliver when patient initiates a breath, [2, 28] cmH2O
Expiratory Trigger Sensitivity—Percentage of peak inspiratory flow that triggers
expiration on patient triggered breath, [5, 70] percent
Rate—Backup/apnea rate to ensure patient is breathing, [4, 33] breaths per minute
Flow Trigger—Trigger for patient breath detection; used for timing breath intervals and
timing delivery of Psupport pressure, [1.0, 20.0] liters per minute
Oxygen—What percentage of oxygen in the air to deliver, [20, 100] percent.
Operation Sequence
1. Set motor to spin at calculated RPM to achieve PEEP setting pressure in the airway
2. Each time the flow sensor is read, activate the flow trigger if the measured value is higher than
or equal to flow trigger setting
3. When the flow trigger is activated, record patient breath occurring. Calculate breaths per
minute from this record and activate breaths per minute alarm if the value is out of alarm upper
or lower limits. Activate support breath
4. Set motor to spin at calculated RPM to achieve PEEP + Psupport pressure in the airway
5. Measure airway flow until value reaches Expiratory Trigger Sensitivity calculated flow value
6. Set motor to spin at calculated RPM to achieve PEEP setting pressure in the airway
6. Pressure Controlled Continuous Mandatory Ventilation (P-CMV) Mode
Delivers a set amount of time cycled pressure controlled breaths and assists spontaneous
breathing activity with full mechanical breaths
Settings
PEEP—Constant pressure to maintain while not in inspiration, [2, 28] cmH2O
Pcontrol—High pressure value to deliver during inspiration, [2, 28] cmH2O. This setting
means breaths are pressure controlled.
Rate—Set breath rate, used with I:E ratio to time cycle breaths, [4, 33] breaths per minute
I:E Ratio—Inspiratory to Expiratory ratio, used with breath rate to time cycle breath
delivery, [1:8, 8:1]
Flow Trigger—Trigger for patient breath detection; used for timing breath intervals and
timing delivery of Psupport pressure, [1,0, 20.0] liters per minute
Oxygen—What percentage of oxygen in the air to deliver, [20, 100] %.
Operation Sequence
1. Achieve PEEP pressure in airway
2. Calculate time of inspiration and time of exhalation with rate and I:E ratio
3. Achieve PEEP + Pcontrol pressure in airway and wait during time of inspirtion (e.g., a
number of seconds), then achieve PEEP pressure in airway and wait during time of exhalation
(e.g., a number of seconds)
4. When flow trigger is activated, deliver a full mechanical breath (PEEP + Pcontrol) and re-time
the next breath so as to keep the set breath rate
7. Pressure Controlled Synchronized intermittent Mandatory Ventilation (P-SIMV)
Delivers a set amount of time cycled pressure controlled breaths and assists spontaneous
breathing activity with full mechanical breaths up to the guaranteed rate of breaths then with
support breaths
Settings
PEEP—Constant pressure to maintain while not in inspiration, [2, 28] cmH2O
Pcontrol—High pressure value to deliver during inspiration, [2, 28] cmH2O. The setting
means breaths are pressure controlled
Psupport—Pressure to deliver when patient initiates a breath at more than the set rate, [2,
28] cmH2O
Expiratory Trigger Sensitivity—Percentage of peak inspiratory flow that triggers
expiration on patient triggered support breath, [5, 70] percent
Rate—Set breath rate, used with I:E ratio to time cycle breaths, [4, 33] breaths per minute
I:E Ratio—Inspiratory to Expiratory ratio, used with breath rate to time cycle breath
delivery, [1:8, 8:1]
Flow Trigger—Trigger for patient breath detection; used for timing breath intervals and
timing delivery of Psupport pressure, [1.0, 20.0] liters per minute
Oxygen—What percentage of oxygen in the air to deliver, [20, 100] percent.
Operation Sequence
1. Achieve PEEP pressure in airway
2. Calculate time of inspiration and time of exhalation with breath rate and I:E ratio
3. Achieve PEEP + Pcontrol pressure in airway and wait during time of inspiration (e.g., a
number of seconds), then achieve PEEP pressure in airway and wait during time of exhalation
(e.g., a number of seconds)
4. When the flow trigger is activated, deliver a full mechanical breath (PEEP + Pcontrol) if the
breath is close to matching the set breath rate (up to 60/rate breaths per minute). If more breaths
are triggered they will be support breaths (PEEP + Psupport pressure, using expiratory trigger
sensitivity to cycle the breath)
8. Volume Controlled Continuous Mandatory Ventilation (V-CMV) Mode
Delivers a set amount of time cycled volume controlled breaths and assists spontaneous
breathing activity with full mechanical breaths
Settings
PEEP—Constant pressure to maintain while not in inspiration, [2, 28]0 cmH2O
Vt~Volume of air to deliver with each breath [25, 2000]milliliters. This setting means
breaths are volume controlled
Rate—Set breath rate, used with I:E ratio to time cycle breaths, [4, 33] breaths per minute
I:E Ratio—Inspiratory to Expiratory ratio, used with breath rate to time cycle breath
delivery, [1:8, 8:1]
Flow Trigger—Trigger for patient breath detection; used for timing breath intervals and
timing delivery of Psupport pressure, [1.0, 20.0] liters per minute
Oxygen—What percentage of oxygen in the air to deliver, [20, 100] %.
Operation Sequence
1. Achieve PEEP pressure in airway
2. Calculate time of inspiration and time of exhalation with rate and I:E ratio, and calculate flow
required to deliver an amount of air (e.g., a number of milliliters according to the Vt setting)
during inspiration time (e.g., a number of seconds).
3. Achieve calculated flow into airway and wait during time of inspiration ,e.g., a number of
seconds.), then achieve PEEP pressure in airway and wait during time of exhalation (e.g., a
number of seconds)
4. When flow trigger is activated, deliver a full mechanical breath (using calculated flow and
inspiration time) and re-time the next breath so as to keep the set breath rate
9. Volume Controlled Synchronized Intermittent Mandatory Ventilation (V-SIMV)
Delivers a set amount of time cycled volume controlled breaths and assists spontaneous
breathing activity with full mechanical breaths up to the set rate of breaths then with support
breaths
Settings
PEEP—Constant pressure to maintain while not in inspiration, [2, 28] cmH2O
Vt—Volume of air to deliver with each breath [25, 2000]cmH2O. This setting means
breaths are volume controlled
Psupport—Pressure to deliver when patient initiates a breath at more than the set rate, [2,
28] cmH2O
Expiratory Trigger Sensitivity—Percentage of peak inspiratory flow that triggers
expiration on patient triggered support breath, [5, 70] percent
Rate—Set breath rate, used with I:E ratio to time cycle breaths, [4, 33] breaths per minute
I:E Ratio—Inspiratory to Expiratory ratio, used with breath rate to time cycle breath
delivery, [1:8, 8:1]
Flow Trigger—Trigger for patient breath detection; used for timing breath intervals and
timing delivery of Psupport pressure, [1.0, 20.0] liters per minute
Oxygen—What percentage of oxygen in the air to deliver, [20, 100] percent.
Operation Sequence
1. Achieve PEEP pressure in airway
2. Calculate time of inspiration and time of exhalation with breath rate and I:E ratio, and
calculate flow requirements to deliver an amount of air (e.g., a number of milliliters according to
the Vt setting) during inspiration time (e.g., a number of seconds)
3. Achieve the calculated flow into airway and wait during time of inspiration (e.g., a number of
seconds), then achieve PEEP pressure in airway and wait during time of exhalation (e.g., a
number of seconds)
4. When the flow trigger is activated, deliver a full mechanical breath (using calculated flow and
inspiration time) if the breath is close to matching the set breath rate (up to 60/rate breaths per
minute). If more breaths are triggered they will be support breaths (PEEP + Psupport pressure,
using expiratory trigger sensitivity to cycle the breath)
10. User Custom Modes
User can combine any number of settings available to the predefined modes into their
own custom mode, combined with custom alarm settings and which measured values to
display/emphasize/graph
Depending on the settings chosen, the system will set up the custom mode according to
each setting and each setting's value.
The system may prevent custom modes from being controlled by more than one source
(e.g., if the mode is pressure controlled, it cannot also be volume controlled).

It will be appreciated that certain parameters having different names in different modes (e.g., IPAP and Phigh, or EPAP and Plow) mentioned in Table 1 above may be referring to similar or the same concepts but are differently named in the respective different modes.

Additionally, in order to facilitate the operations in the foregoing exemplary modes and to provide relevant information to a user, the ventilator system may utilize a plurality of sensors (e.g., as discussed above in connection with FIGS. 1-4) to determine various measurements. A table setting forth exemplary parameters that may be measured and utilized by the ventilator system is provided below.

TABLE 2
Exemplary Measurements

Flow (I)—Displayed (Default)—provides an indication of how much air is being provided per
breath (useful information for a healthcare provider and also usable as feedback for blower
control)
Inspiratory Flow
Direct output of flow sensor on inspiratory path, liters per minute
Can be displayed in waveform in GUI
No default alarms
FiO2—Displayed (Default)—provides linkage between oxygen being provided and oxygen
being delivered to the patient (useful information for a healthcare provider)
Fraction of Inspired Oxygen
Direct output of oxygen sensor on inspiratory path, percent
Default alarm limits: High 100, Low 70
Pinsp—Displayed (Default)—provides a measurement of the force with which the air is being
delivered (useful information for a healthcare provider for optimizing patient treatment)
Inspiratory Pressure
Direct output of pressure sensor on inspiratory path, cmH2O
Can be displayed in waveform in GUI
Default alarm limits: High 40, Low 2
Ftotal—Displayed (Default)—provides a measurement of breath rate (useful information for a
healthcare provider)
Measured breath rate (aka frequency)
Measured as average number of breath initiations per minute including spontaneous,
supported, and fully mechanical breaths
Default alarm limits: High 30, Low 3
VTE—Displayed (Default)—provides a measurement of how air is being exhaled (useful
information for a healthcare provider to prevent “breath stacking” where there is too much
residual pressure in the lungs)
Expiratory Tidal Volume
Measured as integration of expiratory flow sensor over time of expiration (for applicable
modes), milliliters
Can be displayed in waveform in GUI
Default alarm limits: High 850, Low 250
PetCO2—Displayed (Default)—provides a measurement for understanding the balance between
O2 and CO2 in patient (useful information for a healthcare provider)
Percent end tidal CO2
Measured from volatile compound sensor and calculation with atmospheric pressure,
millimeters of mercury
Default alarm limits: High 60, Low 30
ExpMinVol—Displayed (Default)—provides indications of lung health and function (useful
information for a healthcare provider)
Expiratory Minute Volume
Average volume of expiration over a 1 minute interval, Liters per minute
Measured as calculation of measured breath rate and VTE
Ppeak—Displayed (Default)—provides a measurement of peak pressure (useful information for
a healthcare provider to ensure lung safety and avoid over-inflation of the lungs)
Peak Pressure
Maximum pressure achieved in inspiratory phase of breath, cmH2O
Used to alter motor RPM calculations for each mode
Flow (E)—Not Displayed (Default)—provides a measurement of expiratory flow (useful
information for a healthcare provider to understand how a patient's physiology and musculature
is responding to removal of air from lungs)
Expiratory Flow
Direct output of flow sensor on expiratory path, liters per minute
Can be displayed in waveform in GUI
No default alarms
Pexp—Not Displayed (Default)—provides a measurement of expiratory pressure (useful
information for a healthcare provider to understand how a patient's physiology and musculature
is responding to removal of air from lungs)
Expiratory Pressure
Direct output of pressure sensor on expiratory path, cm.H2O
Can be displayed in waveform in GUI
Default alarm limits: High 40, Low 2
Flow (P)—Not Displayed (Default)—provides a measurement of proximal flow, which is a
relatively more precise measurement of volume of air delivered during a breath (useful
information for a healthcare provider, especially for pediatric patients)
Proximal Flow
Direct output of flow sensor connecting patient face apparatus to ventilator breathing
circuit, liters per minute
Can be displayed in waveform in GUI
No default alarms

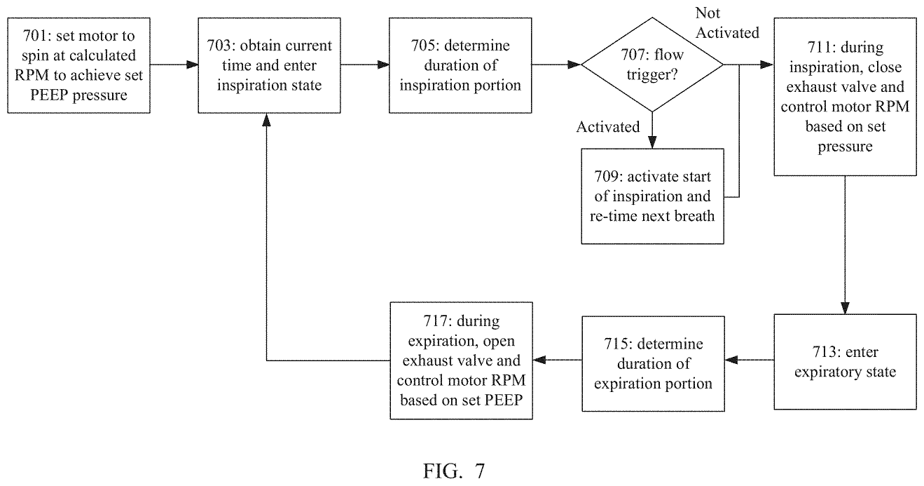

FIG. 7 is an exemplary flowchart depicting a process for performing breath loops in a P-CMV mode of a software-defined ventilator system according to an exemplary embodiment. As mentioned above, the configurable settings for the P-CMV mode may include PEEP, Pcontrol, Rate, I:E Ratio, Flow Trigger, and Oxygen.

At stage 701, upon starting ventilation for the P-CMV mode, the controller may control a motor to spin at a calculated RPM to achieve a set PEEP pressure.

At stage 703, after PEEP pressure is achieved, the controller obtains a current time and enters an inspiration state.

At stage 705, the controller determines a duration for the inspiration portion of the breath loop. The calculated duration may be based on the breath rate setting and the I:E ratio (for example, at a breath rate of 10 breaths per minute, each breathing loop is 6 seconds, and at an I:E ratio of 2:1, the inspiration portion would be 4 seconds and the expiration portion would be 2 seconds).

At stage 707, the controller obtains measurements from a flow sensor and determines whether to activate a flow trigger. If the flow trigger is not activated, then the controller proceeds to stage 711 and performs inspiration for the calculated duration, wherein the exhaust valve on the expiratory channel is closed for the duration of the inspiration. If the flow trigger is activated, then the controller proceeds to stage 709 prior to stage 711. At stage 709, the start of inspiration is activated and a next breath is re-timed to meet the breath rate setting. The flow trigger senses whether a patient is initiating a breath, and then provides pressure and flow to help the patient complete the breath. This is advantageous both for invasive and non-invasive situations. For example, if the ventilator system is providing full lung function to a sedated patient, and in certain situations (e.g., when the patient is being revived), the flow trigger helps the patient to take over breathing but still with assistance from the ventilator system. In a non-invasive application, if after a certain time the patient is not taking a breath, the machine takes over and breathes for the patient, but the flow trigger helps to encourage the patient to take over the breathing function.

At stage 711, during the inspiration, the controller sets the motor RPM based on the Pcontrol setting to achieve a set PEEP+Pcontrol pressure. If there's a difference between the measured pressure and the set pressure, then the motor RPM is adjusted to meet the set pressure.

At stage 713, upon the inspiration duration being complete, the controller enters the expiratory state.

At stage 715, in the expiratory state, the controller determines a duration for the expiration portion of the breath loop (e.g., using the breath rate setting and the I:E ratio).

At stage 717, during the expiration portion of the breath loop, the exhaust valve on the expiratory channel is opened and the motor is set to spin at a calculated RPM for achieving the set PEEP pressure. If there's a difference between the measured pressure and the set pressure, then the motor RPM is adjusted to meet the set pressure.

Upon the expiration duration being complete, the controller proceeds back to stage 703 and re-enters the inspiration state, and another breath loop may be carried out.

It will be appreciated that the exemplary process depicted in FIG. 7 is merely illustrative, and that exemplary embodiments of the present application are not limited to the implementation details shown in FIG. 7. For example, the inspiration and expiration durations may be calculated at the outset and may not need to be re-calculated for each breath loop; instead, the inspiration and expiration durations may be re-calculated based on there being a change to the breath loop or I:E settings.

It will be appreciated that the execution of the various machine-implemented processes and steps described herein may occur via the execution, by one or more respective processors, of processor-executable instructions stored on a tangible, non-transitory computer-readable medium, such as random access memory (RAM), read-only memory (ROM), programmable read-only memory (PROM), and/or another electronic memory mechanism.

While embodiments of the invention have been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of“or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A ventilator system, comprising:
a control system comprising a controller and a user interface; and
a pneumatic system comprising an inspiratory channel and an expiratory channel;
wherein the inspiratory channel further comprises a blower connected to an oxygen source, wherein the blower is configured to be controlled by the controller, and to deliver oxygen and/or air to a patient via the inspiratory channel;
wherein the expiratory channel includes an exhaust valve configured to be controlled by the controller and an outlet;
wherein the pneumatic system further includes a plurality of sensors configured to provide oxygen measurements, pressure measurements, and flow measurements;
wherein the controller is configured to receive measurements from the plurality of sensors and to cause the measurements to be displayed via the user interface; and
wherein the controller is configured to receive a user input via the user interface for controlling a treatment regimen for the patient, and to control the blower and the exhaust valve according to the treatment regimen.

2. The ventilator system according to claim 1, wherein the pneumatic system further comprises:
a negative pressure channel comprising a second blower and a second exhaust, wherein the second blower is configured to blow air out through the second exhaust to maintain a negative pressure environment proximate to a patient's face or head.

3. The ventilator system according to claim 2, wherein the pneumatic system further includes a biofilter disposed in the negative pressure channel for blocking transmission of viral or bacterial particles.

4. The ventilator system according to claim 1, wherein the pneumatic system further includes a motor controller/driver connected to the blower, and wherein the controller is configured to communicate with the motor controller/driver to set a revolution rate for a motor of the blower.

5. The ventilator system according to claim 1, wherein the plurality of sensors include an oxygen sensor disposed in the inspiratory channel, one or more pressure sensors disposed in the inspiratory and/or expiratory channels, a first flow meter disposed in the inspiratory channel, and a second flow meter disposed in the expiratory channel.

6. The ventilator system according to claim 1, wherein the plurality of sensors a first combined flow/pressure sensing device in the inspiratory channel and a second combined flow/pressure sensing device in the expiratory channel.

7. The ventilator system according to claim 1, wherein the plurality of sensors further include a carbon dioxide sensor disposed in the expiratory channel.

8. The ventilator system according to claim 1, wherein the pneumatic system further includes biofilters disposed in the inspiratory and expiratory channels for blocking transmission of viral or bacterial particles.

9. The ventilator system according to claim 1, wherein the inspiratory channel further includes an inspiratory hold valve; and
wherein the controller is configured to control the exhaust valve and the inspiratory hold valve to perform an inspiratory hold operation, wherein during the inspiratory hold operation, both the exhaust valve and the inspiratory hold valve are closed.

10. The ventilator system according to claim 1, wherein the controller is configured to communicate with a nurse call system.

11. The ventilator system according to claim 10, wherein the controller is configured to trigger a nurse call to the nurse call system based on determining that one or more measurements from the plurality of sensors satisfies nurse call criteria.

12. The system according to claim 1, wherein the controller is configured to communicate over a communication network and to be remotely controlled by a healthcare provider device via the communication network.

13. The system according to claim 1, wherein the user interface includes a video camera, and wherein the controller is configured to provide telemedicine communications between a patient and a healthcare provider via the video camera.

14. The system according to claim 1, wherein the controller is configured to obtain flow measurements from a proximal flow sensor disposed in a tubing circuit connected to the pneumatic system, wherein the proximal flow sensor is located proximate to a patient's face or airway.

15. The system according to claim 1, wherein the controller is configured to operate the pneumatic system in a plurality of modes corresponding to respective treatment regimens, wherein each mode comprises a respective set of adjustable parameters.

16. The system according to claim 15, wherein the plurality of modes includes a continuous positive airway pressure (CPAP) mode, a biphasic positive airway pressure (BiPAP) mode, a pressure controlled inverse ratio ventilation (PC-IRV) mode, an airway pressure release ventilation (APRV) mode, a spontaneous (SPONT) mode, a pressure controlled continuous mandatory ventilation (P-CMV) mode, a pressure controlled synchronized intermittent mandatory ventilation (P-STMV) mode, a volume controlled continuous mandatory ventilation (V-CMV) mode, and a volume controlled synchronized intermittent mandatory ventilation (V-SIMV) mode.

17. A ventilator system, comprising:
a control system comprising a controller and a user interface; and
a pneumatic system comprising an inspiratory channel and an expiratory channel;
wherein the inspiratory channel further comprises a blower connected to an oxygen source, wherein the blower is configured to be controlled by the controller, and to deliver oxygen and/or air to a patient via the inspiratory channel;
wherein the expiratory channel includes an exhaust valve configured to be controlled by the controller and an outlet;
wherein the pneumatic system further includes a plurality of sensors configured to provide oxygen measurements, pressure measurements, and flow measurements;
wherein the controller is configured to operate the pneumatic system in a plurality of modes corresponding to respective treatment regimens, wherein each mode comprises a respective set of adjustable parameters.

18. The ventilator system according to claim 17, wherein the controller is further configured to automatically adjust respective parameters based on measurements from the plurality of sensors.

19. The ventilator system according to claim 17, wherein the controller is configured to adjust at least the following parameters:
continuous positive airway pressure;
breath rate;
flow trigger;
oxygen percentage;
inspiratory pressure to deliver;
expiratory pressure to deliver;
expiratory to inspiratory ratio or inspiratory to expiratory ratio;
positive expiratory end pressure (PEEP);
high pressure value to deliver during inspiration;
pressure to deliver when the patient initiates a breath;
expiratory trigger sensitivity; and
volume of air to deliver with each breath.

20. The ventilator system according to claim 19, wherein the controller is configured to adjust a respective parameter with a respective mode according to a respective value range corresponding to the respective parameter for the respective mode.
